(19) United States
(12) Patent Application Publication
(10) Pub. No.: US 2020/0179596 A1
(43) Pub. Date: Jun. 11, 2020

(54) Fill-Finish Carriers For Drug Containers

(71) Applicant: UNL Holdings LLC, New York, NY (US)

(72) Inventors: Alexis Marie Adolphe Dechelette, Lancaster, PA (US); Shaun R. Devitt, Wayne, PA (US); Robert G. Jeter, Jr., Glen Rock, PA (US); Lawton Laurence, Phoenixville, PA (US); Mark A. Destefano, Collegeville, PA (US)

(21) Appl. No.: 16/615,701

(22) PCT Filed: Jun. 6, 2017
(86) PCT No.: PCT/IB2017/000836

§ 371 Date: Nov. 21, 2019

(51) Int. Cl. A61M 5/162

(52) U.S. Cl. CPC A61M 5/162; A61M 2205/50; A61M 2209/045; A61M 2205/13

(57) ABSTRACT

A fill-finish cartridge includes a carrier, a fluid pathway connection, a needle insertion mechanism, and a drug container. The cartridges enable drug containers to be filled with pharmaceutical treatments using standard filling equipment and process systems, while maintaining the sterility and container integrity of the fluid pathway. The fill-finish cartridges of the present invention can be nested or removably housed in fill-finish trays for batch filling in standard operating processes. As such, the adaptable fillfinish cartridges of the present invention may be flexibly inserted, attached, mounted, or otherwise removably positioned in fill-finish trays. These embodiments, accordingly, may provide novel and cost-efficient assemblies and cartridges which are readily integrated into drug filling processes. Methods of assembly, manufacture, and use are also provided.

### CROSS-REFERENCE TO RELATED APPLICATIONS

This application claims priority to U.S. Provisional Application Nos. 62/346,194 filed on Jun. 6, 2016 and 62/372,165 filed on Aug. 8, 2016, which are included by reference herein in their entirety for all purposes.

### FIELD OF THE INVENTION

The present invention relates to fill-finish cartridges for use in pharmaceutical fill-finish processes. More specifically, the embodiments of the present invention relate to fill-finish cartridges that include carriers that enable drug containers to be filled and finished in standard fill-finish processes. Methods for manufacturing and filling such cartridges, and their methods of use, are also provided.

### BACKGROUND OF THE INVENTION

Parenteral delivery of various drugs, i.e., delivery by means other than through the digestive track, has become a desired method of drug delivery for a number of reasons. This form of drug delivery by injection may enhance the effect of the substance being delivered and ensure that the unaltered medicine reaches its intended site at a significant concentration. Similarly, undesired side effects associated with other routes of delivery, such as systemic toxicity, can potentially be avoided through parenteral delivery. By bypassing the digestive system of a mammalian patient, one can avoid degradation of the active ingredients caused by the catalytic enzymes in the digestive tract and liver and ensure that a necessary amount of drug, at a desired concentration, reaches the targeted site.

Traditionally, manually operated syringes and injection pens have been employed for delivering parenteral drugs to a patient. More recently, parenteral delivery of liquid medicines into the body has been accomplished by administering bolus injections using a needle and reservoir, continuously by gravity driven dispensers, or via transdermal patch technologies. Bolus injections often imperfectly match the clinical needs of the patient, and usually require larger individual doses than are desired at the specific time they are given. Continuous delivery of medicine through gravity-feed systems compromises the patient's mobility and lifestyle, and limits the therapy to simplistic flow rates and profiles. Another form of drug delivery, transdermal patches, similarly has its restrictions. Transdermal patches often require specific molecular drug structures for efficacy, and the control of the drug administration through a transdermal patch is severely limited.

Ambulatory infusion pumps have been developed for delivering liquid medicaments to a patient. These infusion devices have the ability to offer sophisticated fluid delivery profiles accomplishing bolus requirements, continuous infusion and variable flow rate delivery. These infusion capabilities usually result in better efficacy of the drug and therapy and less toxicity to the patient's system. Currently available ambulatory infusion devices are expensive, difficult to program and prepare for infusion, and tend to be bulky, heavy and very fragile. Filling these devices can be difficult and require the patient to carry both the intended medication as well as filling accessories. The devices often require specialized care, maintenance, and cleaning to assure proper functionality and safety for their intended long-term use, and are not cost-effective for patients or healthcare providers.

As compared to syringes and injection pens, pump type delivery devices can be significantly more convenient to a patient, in that doses of the drug may be calculated and delivered automatically to a patient at any time during the day or night. Furthermore, when used in conjunction with metabolic sensors or monitors, pumps may be automatically controlled to provide appropriate doses of a fluidic medium at appropriate times of need, based on sensed or monitored metabolic levels. As a result, pump type delivery devices have become an important aspect of modern medical treatments of various types of medical conditions, such as diabetes, and the like.

While pump type delivery systems have been utilized to solve a number of patient needs, manually operated syringes and injection pens often remain a preferred choice for drug delivery as they now provide integrated safety features and can easily be read to identify the status of drug delivery and the end of dose dispensing. However, manually operated syringes and injections pens are not universally applicable and are not preferred for delivery of all drugs. There remains a need for an adjustable (and/or programmable) infusion system that is precise and reliable and can offer clinicians and patients a small, low cost, light weight, simple to use alternative for parenteral delivery of liquid medicines.

There is a strong market demand for drug delivery devices which are easy-to-use, cost-efficient, and which include integrated safety features. However, manufacturing of such devices can be cost intensive, which results in higher costs to patients. Much of the manufacturing costs can be attributed to the need to maintain a sterile fluid pathway from the drug container to the needle, prior to introduction of the drug to the patient. Some commercial products seek to maintain the sterility of the device by manufacturing the components in a non-sterile environment and then sterilizing the entire device. A recognized downside of such processes is the need to separately fill the drug container after device sterilization but prior to drug injection, as most pharmaceutical compounds are not capable of withstanding the device sterilization process. Alternatively, the drug delivery device may be manufactured as a pre-filled device, wherein the device is filled with the drug aseptically during assembly. Such manufacturing processes may be costly since the entire process must be kept sterile and because the fill and assembly lines need to be specially-tailored for the device. Accordingly, this adds substantial operating costs to pharmaceutical companies and contract drug-fillers.

Drug delivery devices are generally prepared by molding or shaping the various components and then assembling the components. The assembling steps and other processing operations typically produce a device that subsequently must be cleaned to remove particulates adhering to the surfaces to satisfy cleanliness standards for drug delivery devices. After cleaning, conventional drug delivery devices are packaged and sterilized. Such delivery devices have been classified into several general types. The first type is assembled and placed in sterile packaging which can be shipped with a vial or ampoule of a drug or other injectable solution. The vial or ampoule is generally made of glass or other clear material that does not interfere with the stability of the drug during prolonged storage. The delivery device is filled with the drug or other solution at the point of use and injected into the patient. These devices have the disadvantage of increasing the time and difficulty of filling the device at the point of use with increased possibility of contamination of the delivery device and/or drug solution. There is a further risk of glass particles from the ampoules contaminating the drug solution when the ampoules are opened.

Several of these disadvantages are overcome by providing prefilled delivery devices which can be filled with a suitable drug solution prior to use. Prefilled delivery devices, as the term is known in the art, are devices that are filled by the drug manufacturer and shipped to the health care provider or self-administering patient in a condition that is ready for use. Prefilled delivery devices have the advantage of convenience and ease of application with reduced risk of contamination of the drug solution. Prefilled drug delivery devices are generally assembled and packaged in clean rooms to maintain proper cleanliness levels. The clean rooms are equipped with extensive filter assemblies and air control systems to remove particulates and pyrogens from the air in the room and to prevent particulates and pyrogens from entering the room. The operators and other personnel in the clean room are required to wear appropriate protective garments to reduce contamination of the air and the drug delivery devices being manufactured or assembled. As people and equipment enter and leave the clean room, the risk of contamination and introduction of foreign particulates and pyrogens increases. Various operations are able to form clean and sterile drug delivery devices. However, subsequent handling, filling and printing of the drug delivery device can contaminate the device. It is then necessary to clean and sterilize such conventional drug delivery devices before use. Accordingly, there is a continuing need in the industry for an improved system for manufacturing and assembling clean and sterile medical devices and filling such devices.

### BRIEF SUMMARY OF THE INVENTION

The inventors of the present invention have developed fill-finish cartridges which utilize carriers to enable drug containers to be filled with pharmaceutical treatments using standard filling equipment and systems. This advantage is enabled by the novel fill-finish cartridges of the present invention which function to allow the drug containers to nest, mount, or otherwise be removably inserted into trays for standard fill-finish processes, as discussed further below.

The novel fill-finish cartridges of the present invention are notably able to be adapted to drug containers so that they can be filled with pharmaceutical treatments in standard manufacturing fill-finish process lines, while maintaining the sterility and container integrity of the fluid pathway. The fill-finish cartridges, in connection with the drug containers, of the present invention can be nested or removably housed in fill-finish trays for batch filling in standard operating processes. As such, the adaptable fill-finish cartridges and drug containers of the present invention may be flexibly inserted, attached, mounted, or otherwise removably positioned in fill-finish trays. These embodiments, accordingly, may provide novel and cost-efficient assemblies and cartridges which are readily integrated into drug filling processes.

In a first embodiment, the present invention provides a fill-finish cartridge that includes a drug container to hold a drug fluid prior to initiation of an injection. The drug container may be, for example, a glass vial sealed with a pierceable membrane that may be pierced by a fluid pathway connection upon activation by the user. In at least one embodiment, the drug container is a glass barrel tube having a pierceable membrane seal at a distal end and a plunger seal, such as an elastomeric plunger seal, at a proximal end. Upon activation, the fluid pathway connection may be caused to pierce the drug container, thereby permitting fluid to flow from the container through the connection, a fluid conduit, and a needle insertion mechanism for drug delivery to the patient. The fluid pathway connection may also be comprised of one or more components. In at least one embodiment, the fluid pathway connection includes a means for mounting to the drug container, a means for connecting the fluid conduit to the drug container, and optionally a means for disconnecting the fluid conduit from the drug container. The means for mounting may be, for example, a connection collar. The means for connecting the fluid conduit to the drug container may be, for example, a needle or cannula. The means for disconnecting the fluid conduit may be, for example, a secondary retraction mechanism or a closing flange. The fluid pathway assembly may be mounted into a drug delivery device which may include other components to facilitate the activation of the device and the needle insertion, retraction, and other mechanisms of the fluid pathway assembly and the overall device. For example, the device may include a drive mechanism which connects to the plunger seal of the drug container, to force the drug fluid out of the container, through the connection, the fluid conduit, and the needle insertion mechanism for drug delivery to the patient. A number of different drive mechanisms and other known components may be utilized in this way, as would be appreciated by one having ordinary skill in the art.

A drug or pharmaceutical treatment may be filled into the drug container. For example, the drug container may be configured for a pre-filled drug delivery system. In one such configuration, the drug container would have a pierceable seal at a distal end and a plunger seal at a proximal end. The pierceable seal may be fixedly attached, by glue or other known method of adhesion or connection such as compression fit, to the distal end of the container. The container may then be filled with a desired quantity of drug at the proximal end of the container. After completion of the filling, the plunger seal may be mounted at the proximal end of the container. As would be appreciated by one having ordinary skill in the art, this filling and assembly process may be completed under vacuum and/or a sterile environment to facilitate the aseptic manufacturing of the drug container. These drug containers are configured such that they may readily be manufactured individually, or in a group, as is the case in a tray-based filling process. Integration of the fluid pathway assemblies into such standard fill-finish processes is enabled, at least in part, by the novel fill-finish cartridges of the present invention.

Accordingly, in another embodiment the present invention relates to a fill-finish cartridge which includes a carrier and a drug container. As described in further detail below, the carrier may be one or more pieces such that the carrier is expandable or adjustable. Furthermore, the carrier may include a flange at a proximal end of the drug container. The flange may be a fixed flange or a removable flange. The flange may consist of a number of known materials including, but not limited to, glass and plastic. The carrier functions to retain the drug container in a sterile condition, while allowing for easy integration of the drug container into a standard fill-finish process. The fill-finish cartridges can be integrated into standard trays. For example, these cartridges, assemblies, and containers can be removably mounted into standard filling trays for filling in automated assembly and drug filling lines. The containers can then be filled with a pharmaceutical drug or treatment, and then sealed by insertion of a plunger seal into the proximal end of the drug container.

In yet another embodiment, the present invention relates to a method of assembling a fill-finish cartridge which includes the steps of mounting the carrier to the drug container. The method of manufacturing may further include the steps of filling the drug container from an opening at the proximal end; and then movably sealing the proximal end of the drug container by inserting a plunger seal.

In a further embodiment, the present invention relates to the method of using the fill-finish cartridge, which method includes the steps of: filling the drug container with a pharmaceutical drug; mounting the needle insertion mechanism to a first location of a drug delivery device; mounting the drug container to a second location of the drug delivery device; triggering the fluid pathway connection to pierce a permeable seal at a distal end of the drug container; triggering the needle insertion mechanism to insert a cannula into a patient; activating a drive mechanism to force the pharmaceutical drug out of the drug container and through the primary container connect, a fluid conduit, and the cannula of the needle insertion mechanism for drug dispersal into the patient. Upon completion of drug delivery, the method of use may further include the step of triggering the needle insertion mechanism to retract the cannula from the patient. The cannula may be a rigid needle, a flexible tube cannula, or a number of other known conduits for injection and/or drug delivery.

One embodiment of the present disclosure includes a cartridge for use in a medicament filling and finishing process, the cartridge includes a drug container defining a longitudinal axis, a needle insertion mechanism including a needle; a fluid pathway connector coupled to the drug container; a flexible, sterile fluid conduit fluidly coupled to the needle insertion mechanism and to the fluid pathway connector; and a carrier, the carrier including an upper carrier and a lower carrier; wherein the lower carrier includes one or more retention features engaged with the drug container to removably mechanically couple the drug container, the fluid pathway connector, and the needle insertion mechanism

In at least one embodiment the upper carrier includes a junction configured to allow the separation of a tubular body of the upper carrier from a retention sleeve of the upper carrier. Optionally, the junction includes one or more frangible tabs.

In at least one embodiment, the lower carrier defines a lower carrier cavity, the needle insertion mechanism and the fluid pathway connector disposed at least partially within the lower carrier cavity. Additionally, in at least one embodiment the upper carrier defines an upper carrier cavity, the drug container is disposed at least partially within the upper carrier cavity.

In at least one embodiment, the upper carrier includes one or more connection prongs and the lower carrier includes one or more connection recesses, the connection prongs disposed at least partially within the connection recesses to mechanically couple the upper carrier and the lower carrier. Optionally, the upper carrier is decoupled from the lower carrier by rotation of the upper carrier relative to the lower carrier which releases the connection prongs from the connection recesses.

In at least one embodiment, the lower carrier includes a first lower carrier and a second lower carrier, the first lower carrier being connected to the second lower carrier to removably mechanically couple the fluid pathway connector and the needle insertion mechanism. Optionally, disconnection of the first lower carrier and the second lower carrier mechanically decouples the fluid pathway connector and the needle insertion mechanism.

In one embodiment, the cartridge includes a pierceable seal disposed in a distal end of the drug container. It may also include a fluid contained within the drug container and a plunger seal disposed within the drug container adjacent a proximal opening of the drug container.

In one embodiment, the upper carrier includes a flange at a proximal end of the upper carrier.

In one embodiment, the one or more retention features are engaged with a neck of the drug container. Alternatively, the one or more retention features are engaged with a crimp collar of the drug container.

In another embodiment, the cartridge includes a drug container defining a longitudinal axis, a needle insertion mechanism including a needle; a fluid pathway connector coupled to the drug container; a flexible, sterile fluid conduit fluidly coupled to the needle insertion mechanism and to the fluid pathway connector; and a carrier, the carrier including an upper carrier and a lower carrier; wherein the drug container, the fluid pathway connector, and the needle insertion mechanism are removably mechanically coupled by the carrier and wherein the upper carrier includes a junction configured to allow the separation of a tubular body of the upper carrier from a retention sleeve of the upper carrier.

The present disclosure also includes a method of constructing a cartridge for a medicament filling and finishing process, the method including the steps of: fluidly coupling a fluid pathway connector to a needle insertion mechanism; removably mechanically coupling the fluid pathway connector and the needle insertion mechanism by disposing the fluid pathway connector and the needle insertion mechanism in a lower carrier cavity of a lower carrier; connecting an upper carrier to the lower carrier; and disposing a drug container within an upper carrier cavity of the upper carrier and engaging the drug container with one or more retention features of the lower carrier.

The method of constructing may also include connecting a first portion of the lower carrier and a second portion of the lower carrier. It may also include engaging one or more connection prongs of the upper carrier with one or more intermediate recesses of the lower carrier. The method may also include filling the drug container with a fluid. The method may also include the step of placing a plunger seal within the drug container adjacent a proximal opening of the drug container. The method may also include engaging a neck of the drug container with the one or more retention features of the lower carrier. The method may also include engaging a crimp cap of the drug container with the one or more retention features of the lower carrier.

Another embodiment of the present invention includes a method of deconstructing a cartridge for a medicament filling and finishing process, the method including the steps of: decoupling a tubular body of the upper carrier from a retention sleeve of the upper carrier; disconnecting the retention sleeve of the upper carrier from a lower carrier; and mechanically decoupling a fluid pathway connector from a needle insertion mechanism by removing the fluid pathway connector and the needle insertion mechanism from a lower carrier cavity of the lower carrier.

The method of deconstructing may also include rotation of the upper carrier relative to the lower carrier. The rotation of the upper carrier relative to the lower carrier may disengage one or more connection prongs of the upper carrier from one or more connection recesses of the lower carrier. In one embodiment, the method includes separation of a first portion of a lower carrier from a second portion of a lower carrier.

Throughout this specification, unless otherwise indicated, “comprise,” “comprises,” and “comprising,” or related terms such as “includes” or “consists of,” are used inclusively rather than exclusively, so that a stated integer or group of integers may include one or more other non-stated integers or groups of integers. As will be described further below, the embodiments of the present invention may include one or more additional components which may be considered standard components in the industry of medical devices. The components, and the embodiments containing such components, are within the contemplation of the present invention and are to be understood as falling within the breadth and scope of the present invention.

### BRIEF DESCRIPTION OF THE DRAWINGS

The following non-limiting embodiments of the invention are described herein with reference to the following drawings, wherein:

FIG. 1 is an isometric view of a drug delivery device incorporating an embodiment of a drug container and fluid pathway of the present invention;

FIG. 2 is a schematic representation of an exemplary fill-finish cartridge of the present invention;

FIG. 3 is an exploded isometric view of a fill-finish cartridge, according to an embodiment of the invention;

FIG. 4A and FIG. 4B are cross-sectional and elevational views, respectively, of a lower assembly of a fill-finish cartridge according to at least one embodiment;

FIG. 5A and FIG. 5B are cross-sectional and elevational views, respectively, of a fill-finish cartridge according to at least one embodiment of an upper carrier connected to the lower assembly;

FIG. 6A and FIG. 6B are cross-sectional and elevational views, respectively, of a fill-finish cartridge according to at least one embodiment with a drug container disposed within the upper carrier;

FIG. 7A and FIG. 7B are cross-sectional and elevational views, respectively, of a fill-finish cartridge according to at least one embodiment with the upper carrier in a locked configuration;

FIG. 8A and FIG. 8B are cross-sectional and elevational views, respectively, of a fill-finish cartridge according to at least one embodiment which has been filled with a medicament and a plunger seal has been positioned within the drug container;

FIG. 9A and FIG. 9B are cross-sectional and elevational views, respectively, of a fill-finish cartridge according to at least one embodiment wherein a tubular body of the upper carrier has been removed;

FIG. 10A and FIG. 10B are cross-sectional and elevational views, respectively, of a fill-finish cartridge according to at least one embodiment wherein a retention sleeve of the upper carrier has been removed;

FIG. 11 is a cross sectional view of a fill-finish cartridge according to at least one embodiment wherein the lower carrier has been removed;

FIGS. 12A, 12B, and 12C are front, side, and isometric views, respectively, of a lower carrier according to at least one embodiment;

FIG. 13 is an elevational view of an upper carrier according to at least one embodiment;

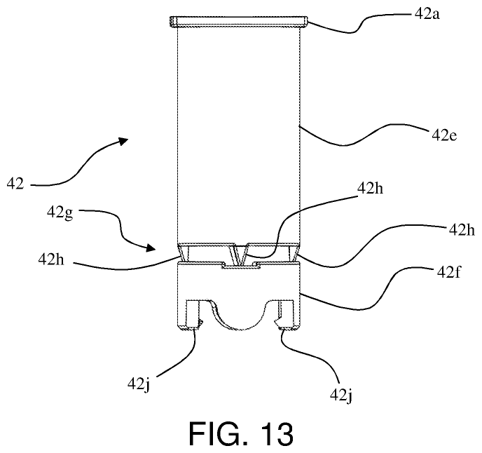

FIG. 14 is an isometric view of an array of fill-finish cartridges according to the present invention, disposed in a fill-finish tub.

### DETAILED DESCRIPTION OF EMBODIMENTS

The inventors of the present invention have developed a cartridge carrier that can enable drug containers to be filled with pharmaceutical treatments using standard filling equipment and systems. This advantage is enabled by the novel fill-finish cartridges of the present invention which function to allow the drug containers to nest, mount, or otherwise be removably inserted into trays for standard fill-finish processes. As such, the adaptable fill-finish cartridges and drug containers of the present invention may be flexibly inserted, attached, mounted, or otherwise removably positioned in fill-finish trays. These embodiments, accordingly, may provide novel and cost-efficient assemblies and cartridges which are readily integrated into drug filling processes. The embodiments of the present invention may be integrated into advanced drug delivery devices, such as injection and/or infusion pumps, which require sterile fluid pathways.

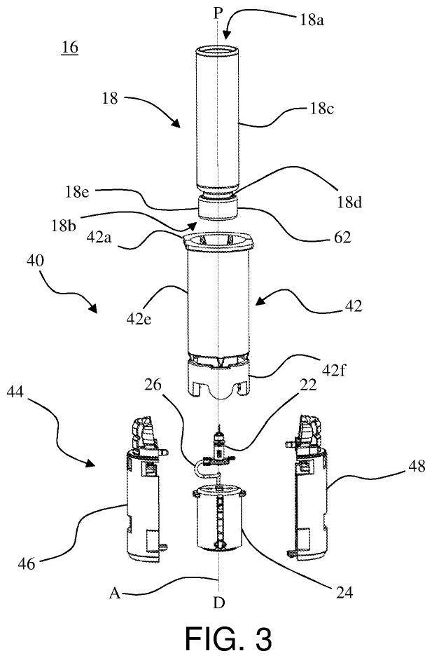

As used herein to describe the fluid pathway assemblies, fill-finish cartridges, drug delivery devices, or any of the relative positions of the components of the present invention, the terms “axial” or “axially” refer generally to a longitudinal axis “A” around which the drug container is preferably formed although not necessarily symmetrically there-around. The term “radial” refers generally to a direction normal to axis A. The terms “proximal,” “rear,” “rearward,” “back,” or “backward” refer generally to an axial direction in the direction labeled “P” in FIG. 3. The terms “distal,” “front,” “frontward,” “depressed,” or “forward” refer generally to an axial direction in the direction labeled “D” in FIG. 3. As used herein, the term “glass” should be understood to include other similarly non-reactive materials suitable for use in a pharmaceutical grade application that would normally require glass. The term “plastic” may include both thermoplastic and thermosetting polymers. Thermoplastic polymers can be re-softened to their original condition by heat; thermosetting polymers cannot. As used herein, the term “plastic” refers primarily to moldable thermoplastic polymers such as, for example, polyethylene and polypropylene, or an acrylic resin, that also typically contain other ingredients such as curatives, fillers, reinforcing agents, colorants, and/or plasticizers, etc., and that can be formed or molded under heat and pressure. As used herein, the term “elastomer,” “elastomeric” or “elastomeric material” refers primarily to cross-linked thermosetting rubbery polymers that are more easily deformable than plastics but that are approved for use with pharmaceutical grade fluids and are not readily susceptible to leaching or gas migration. As used herein, the term “fluid” refers primarily to liquids, but can also include suspensions of solids dispersed in liquids, and gasses dissolved in or otherwise present together within liquids inside the fluid-containing portions of syringes.

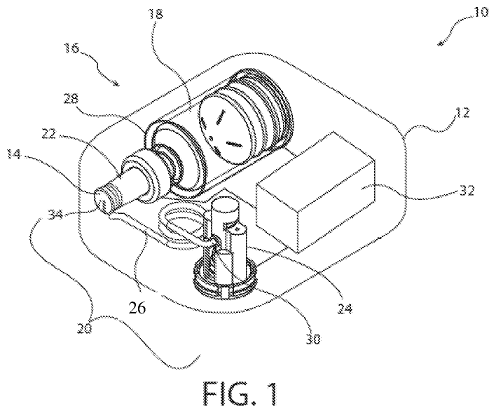

Turning to FIG. 1, there is illustrated a schematic representation of an example of a drug delivery device 10 incorporating aspects of the invention. The device 10 includes a housing 12 having an activation mechanism 14. For ease of understanding, the housing 12 is shown schematically. In accordance with the invention, the device further includes a drug container 18. A fluid pathway assembly 20 may include further structure that facilitates disposition of various components, including, for example, a fluid conduit 26. The fluid pathway connection 22 is disposed substantially adjacent a distal end 28 of the drug container 18, and the needle insertion mechanism 24 is disposed substantially adjacent a distal end 30 of the fluid pathway connection 22. In the illustrated embodiment, the drug container 18 is generally horizontally positioned and perpendicular from a vertically positioned needle insertion mechanism 24. It will be appreciated, however, that the components may be positioned in any appropriate manner

Administration of a drug contained in the drug container 18 may be initiated by the activation mechanism 14. The activation mechanism 14 may include, for example, activation mechanisms that are manually actuated by a user, or that are automatically actuated by, for example, a power and control module 32 that may include, by way of further example, a microprocessor or other automatic administration arrangement with appropriate connections. In this embodiment, the activation mechanism 14 is a button 34 that may be disposed, for example, along an outer surface of the housing 12, and may be selectively depressed by the user. It will be appreciated that the drug delivery device 10 as well as the activation mechanism 14 may be of any appropriate design.

The power and control module 32 may include a power source, which provides the energy for various electrical components within the drug pump, one or more feedback mechanisms, a microcontroller, a circuit board, one or more conductive pads, and one or more interconnects. Other components commonly used in such electrical systems may also be included, as would be appreciated by one having ordinary skill in the art. The one or more feedback mechanisms may include, for example, audible alarms such as piezo alarms and/or light indicators such as light emitting diodes (LEDs). The microcontroller may be, for example, a microprocessor. The power and control module 32 controls several device interactions with the user and may interface with one or more other components of the drug delivery device 10. In one embodiment, the power and control module 32 may identify when an on-body sensor and/or the activation mechanism 14 have been activated. The power and control module 32 may also interface with a status indicator, which may be a transparent or translucent material which permits light transfer, to provide visual feedback to the user. The power and control module 32 may interface with a drive mechanism and/or the integrated sterile fluid pathway connection and drug container 18 through one or more interconnects to relay status indication, such as activation, drug delivery, and/or end-of-dose, to the user. Such status indication may be presented to the user via tactile feedback, such as vibration; auditory tones, such as through the audible alarms; and/or via visual indicators, such as through the LEDs. In a preferred embodiment, the control interfaces between the power and control system and the other components of the drug pump are not engaged or connected until activation by the user. This is a desirable safety feature that prevents accidental operation of the drug pump and may also maintain the energy stored in the power source during storage, transport, and the like.

The power and control module 32 may be configured to provide a number of different status indicators to the user. For example, the power and control module 32 may be configured such that after the on-body sensor and/or trigger mechanism have been pressed, the power and control module 32 provides a ready-to-start status signal via the status indicator if device start-up checks provide no errors. After providing the ready-to-start status signal and, in an embodiment with the optional on-body sensor, if the on-body sensor remains in contact with the body of the user, the power and control module 32 will power the drive mechanism to begin delivery of the drug treatment through the integrated sterile fluid pathway connection 22 and sterile fluid conduit 28. In a preferred embodiment of the present invention, the insertion mechanism 24 and the drive mechanism may be caused to activate directly by user operation of the activation mechanism 14. The integrated sterile fluid pathway connection is connected (i.e., the fluid pathway is opened) by the pneumatic and/or hydraulic force of the drug fluid within the drug container 18 created by activation of the drive mechanism, as is detailed further herein. During the drug delivery process, the power and control module 32 is configured to provide a dispensing status signal via the status indicator. After the drug has been administered into the body of the user and after the end of any additional dwell time, to ensure that substantially the entire dose has been delivered to the user, the power and control module 32 may provide an okay-to-remove status signal via the status indicator. This may be independently verified by the user by viewing the drive mechanism and delivery of the drug dose within the drug container through the window 18 of the pump housing 12. Additionally, the power and control module 32 may be configured to provide one or more alert signals via the status indicator, such as for example alerts indicative of fault or operation failure situations.

Other power and control system configurations may be utilized with the novel drug pumps of the present invention. For example, certain activation delays may be utilized during drug delivery. As mentioned above, one such delay optionally included within the system configuration is a dwell time which ensures that substantially the entire drug dose has been delivered before signaling completion to the user. Similarly, activation of the device may require a prolonged depression (i.e., pushing) of the activation mechanism 14 of the drug delivery device 10 prior to drug pump activation. Additionally, the system may include a feature which permits the user to respond to the end-of-dose signals and to deactivate or power-down the drug pump. Such a feature may similarly require a delayed depression of the activation mechanism, to prevent accidental deactivation of the device. Such features provide desirable safety integration and ease-of-use parameters to the drug pumps. An additional safety feature may be integrated into the activation mechanism to prevent partial depression and, therefore, partial activation of the drug pumps. For example, the activation mechanism and/or power and control system may be configured such that the device is either completely off or completely on, to prevent partial activation. Such features are described in further detail hereinafter with regard to other aspects of the novel drug pumps.

When included, the power and control module 32 may include a processor (not shown) and a memory component (not shown). The processor may be microprocessors or other processors as known in the art. In some embodiments the processor may be made up of multiple processors. The processor may execute instructions for generating administration signal and controlling administration of a drug contained in the drug container 18. Such instructions may be read into or incorporated into a computer readable medium, such as the memory component or provided external to the processor. In alternative embodiments, hard-wired circuitry may be used in place of or in combination with software instructions to implement drug administration. Thus, embodiments are not limited to any specific combination of hardware circuitry and software.

The term “computer-readable medium” as used herein refers to any medium or combination of media that participates in providing instructions to a processor for execution. Such a medium may take many forms. The memory component may include any form of computer-readable media as described above. The memory component may include multiple memory components.

The power and control module 32 may be enclosed in a single housing. In alternative embodiments, the power and control module 32 may include a plurality of components operably connected and enclosed in a plurality of housings.

The power and control module 32 may be configured to generate an administration signal as a function of user actuation, preprogrammed actuation or remote actuation. The power and control module 32 may be communicatively coupled to fill-finish cartridge 16, and/or the drug container 18, the fluid pathway connection 22, and/or the needle insertion mechanism 24 individually.

In accordance with an aspect of embodiments of the invention, in the illustrated embodiment, actuation of the activation mechanism 14, here, depression of the button 34, triggers the needle insertion mechanism 24 to inject a needle or cannula into the patient. The integrated sterile fluid pathway connection is connected (i.e., the fluid pathway is opened) by the pneumatic and/or hydraulic force of the drug fluid within the drug container 18 created by activation of the drive mechanism, as is detailed further herein. Thus, actuation of activation mechanism 14 results in the completion of a drug pathway from the drug container 18 through the fluid pathway connection 22, the fluid conduit 26, and the needle insertion mechanism 24 to the patient (not shown). Actuation of the activation mechanism 14 may also result in a drive mechanism acting upon structure associated with the drug container 18 to force fluid through the sterile pathway. In an embodiment of the present invention, the needle insertion mechanism 24 may be triggered to retract the needle from the patient upon completion of drug delivery, giving a clear end of dose delivery indication. The housing 12 may additionally include, for example, a window through which the drug container 18 may be viewed to confirm drug delivery.

The fill-finish cartridges of the present invention consist of a drug container, a fluid pathway connector, and a needle insertion mechanism. These components are maintained in alignment by a carrier, allowing for use of the fill-finish cartridge in a traditional fill-finish process. According to an aspect of embodiments of the invention, the drug container 18 is filled prior to assembly into the housing 12 of the drug delivery device 10. In this regard, the drug container 18 is sufficiently robust to withstand procedures for sterilizing the drug container 18, in some embodiments prior to fill, and in some embodiments after fill. After the sterile filling of the drug container 18 it may be positioned as needed within a drug delivery device 10. Providing a pre-filled device to a user or clinician may simplify the operation of the device and reduce the number of steps of operation. Filling of the drug container prior to assembly into the drug delivery device using the fill-finish cartridges of the present disclosure eliminates the need for terminal sterilization of the entire device. This potentially reduces the cost or size of the device.

According to another aspect of embodiments of the invention, various embodiments of individual components of the fill-finish cartridge 16 may be assembled in various configurations to provide various embodiment of the fill-finish cartridge 16. The following disclosures assigned to the assignee of this disclosure disclose exemplary structures of individual elements that may be incorporated into the fill-finish cartridge 16, and are incorporated herein by reference for everything disclosed therein: U.S. application Ser. No. 13/600,114; U.S. application Ser. No. 13/599,727; U.S. application Ser. No. 13/612,203; U.S. application Ser. No. 13/796,156; U.S. application Ser. No. 14/466,403; U.S. application Ser. No. 15/514, 951; international patent application number PCT/US2016/017534; and international patent application number PCT/US2016/020486.

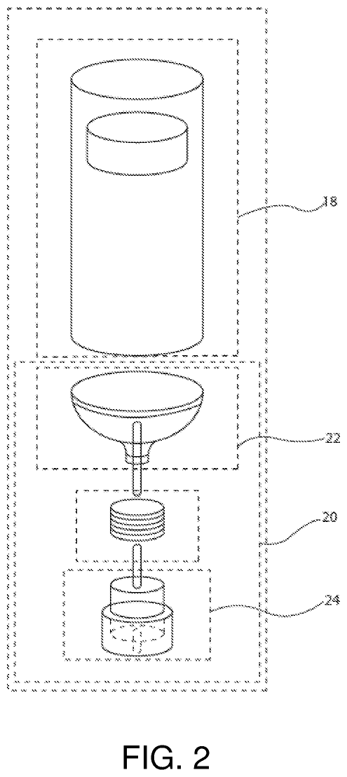

In various embodiments, the fill-finish cartridge 16 may be maintained with the components in axial alignment during the fill-finish process, as well as in use with a drug delivery device 10. That is, for example, the needle insertion mechanism 24 may be disposed axially with the remainder of the fill-finish cartridge 16 during both the fill-finish process, such as shown in FIG. 2, and in use in a drug delivery device. In other embodiments, the fill-finish cartridge 16 may be maintained with the components in axial alignment during the fill-finish process, such as illustrated in FIG. 2, while the components may be maintained in other than axial alignment in use with a drug delivery device 10. For example, as illustrated in FIG. 1, the needle insertion mechanism 24 is disposed spaced apart from the fluid pathway connection 22 and the drug container 18, and at a 90° orientation. In other embodiments, the fill-finish cartridge may be maintained with the components in other than axial alignment during the fill-finish process, yet be axially aligned in use with a drug delivery device 10. In other embodiments, the fill-finish cartridge may be maintained with the components in other than axial alignment during both the fill-finish process and in use with a drug delivery device 10.

Further, a carrier may be provided, as will be explained in more detail below. Such a carrier may be integrated with the structure of the fill-finish cartridge 16 such that it is maintained about or along at least a portion of the fill-finish cartridge 16 in the drug delivery device 10, or such a carrier may be fully or partially disposable. A carrier may perform a number of functions, such as the maintenance of the relative positions of various of the fill-finish cartridge components during assembly, a fill-finish process, or other operations performed on the fill-finish cartridge or a drug delivery device incorporating the same; a carrier or a portion of a carrier may be utilized in the interaction of the fill-finish cartridge with a drug delivery device 10, such as in attachment of the fill-finish cartridge 16 into a drug delivery device 10. More detailed explanations of various examples of such structures in varied configurations follow; it is not the intention to limit the structures to those particular configurations. Rather, the individual arrangements explained are provided as examples of various possible configurations and structures within the purview of this disclosure.

FIG. 3 shows an exploded view of one embodiment of the fill-finish cartridge 16 of the present invention. The fluid pathway assembly 20 includes a needle insertion mechanism 24 coupled to a fluid pathway connection 22 by a fluid conduit 26. A proximal end of the needle insertion mechanism 24 is connected to a distal end of a fluid conduit 26, which is connected at its proximal end to the fluid pathway connection 22.

The needle insertion mechanism 24 may be of any appropriate design so long as it may be sterilized prior to the placement of the fill-finish cartridge 16 in a drug delivery device. Examples of such needle insertion mechanisms 24 for implants and liquid drugs are disclosed in U.S. Applications Ser. Nos. 13/599,727 and 15/514,951 and international application number PCT/US2016/017534, which is assigned to the assignee of this application and is incorporated herein by reference for everything disclosed therein. It will be noted that the needle insertion mechanism 24 of FIG. 3 includes an axial structure, such that the administration needle (not visible in FIG. 3) extends axially from a distal end of the fill-finish cartridge 16 for administration. It will be appreciated, however, that a needle insertion mechanism 24 that is disposed at an angle to an axis of the fluid pathway connection 22 and/or drug container 18 could alternately be utilized.

The fluid pathway connector 22 may also be of any appropriate design so long as it may be sterilized prior to the placement of the fill-finish cartridge 16 in a drug delivery device. Examples of such fluid pathway connectors 22 are disclosed in U.S. applications Ser. Nos. 13/612,203, 13/796,156, 14/466,403 and international application PCT/US2016/020486, which are assigned to the assignee of this application and is incorporated herein by reference for everything disclosed therein. In at least some embodiments, the fluid pathway connector includes a piercing member configured to, upon activation, pierce a pierceable seal of the drug container to establish a fluid flow path from the drug container, through the fluid pathway connector, fluid conduit and to the needle insertion mechanism for delivery to the patient.

The components of the fluid pathway assembly 20, including the needle insertion mechanism 24, the fluid pathway connection 22, and the fluid conduit 26 are formed of materials that may be sterilized by conventional sterilization techniques and machinery. The fluid conduit 26 may be formed of any appropriate material, for example, a length of flexible tubing, such as plastic tubing. It will be appreciated, however, that fluid pathway connection 22 and the needle insertion mechanism 24 may be directly attached in some embodiments (not illustrated).

The components of the fluid pathway assembly 20 may be sterilized in advance of such connections, or may be connected prior to sterilization as a unified component. If sterilized in advance of such connections, the fluid pathway assembly 20 may include an additional seal at the fluid pathway connection 22, such as a permeable seal that may be pierced during assembly or actuation (not illustrated).

The drug container 18 of this and each of the embodiments may be of any appropriate material and of any appropriate shape and size, and may include a seal to maintain the integrity and sterility of a drug contained therein. For example, the drug container 18 may be formed of glass, plastic, or other appropriate material. The drug container 18 of this and each of the embodiments may include structure that facilitates handling, mounting within a drug delivery device, sterilization, and/or interface with other components of the fill-finish cartridge 16. For example, a flange (not shown) may be provided at any appropriate location along the drug container 16. Such a flange may be integrally formed with the drug container 18 or may be a separate element that is secured to the drug container. In the illustrated embodiment, carrier 40 includes flange 42a to facilitate use in a fill-finish tray. The drug container 18 is an elongated, generally annular structure and may include elongated section 18c, neck 18d, and collar 18e. This allows the overall outer diameter of the drug container 18 to be substantially the same as that of the elongated section 18c. This minimizes the amount of space occupied by the drug container 18 in the drug pump and may allow for a smaller overall size of the drug pump. This may lessen the inconvenience to the patient.

In order to facilitate both filling the primary drug delivery container 18 and administering medication from the drug delivery container, the drug container 18 may include openings 18a, 18b at the proximal and distal ends, respectively. In order to seal the drug container 18, a permeable or pierceable seal 50 may be provided at a distal end of the drug container 18. In this way, once filled, a drug contained within the drug container 18 may be maintained in a sterile environment until such time as the seal 50 is pierced by the fluid pathway connection 22 to complete the fluid pathway. The permeable seal 50 may be of any appropriate design and material.

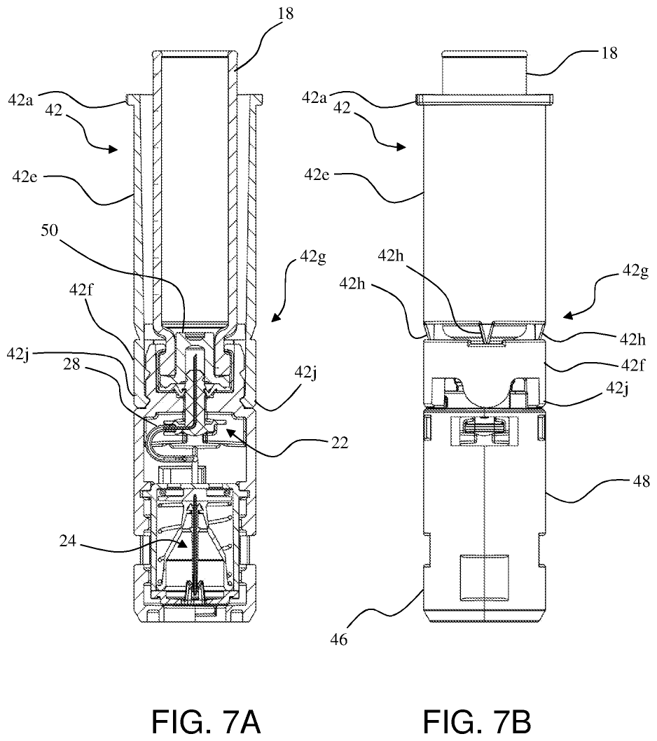

For operational efficiency, the needle insertion mechanism 24 may be coupled to the fluid pathway connection 22, and the fluid pathway connection 22 may be connected to the permeable seal 50 with the needle insertion mechanism 24 maintained in the non-piercing configuration through the sterilization, filling, and assembly processes. In this way, the fill-finish cartridge 16 may appear as shown in FIG. 7B, with the fluid pathway assembly 20 residing substantially hidden from the external environment by the carrier 40. Once the drug container 18 is filled with a pharmaceutical treatment, a plunger seal 19 may be provided in the proximal end of the drug container 18 to provide a closed fill-finish cartridge 16. In the embodiment illustrated, an elastomeric plunger seal 19 is inserted into the proximal end of the drug container 18. It will be appreciated, however, that other appropriate sealing arrangement may be provided.

According to another aspect of the invention, the fluid pathway assemblies may be maintained in a sterile condition and the drug containers of each assembly may be filled with a pharmaceutical compound aseptically using processes similar to those known in the art. After a pharmaceutical treatment is filled into the drug container and the container is sealed, for example with the plunger seal 19, the fill-finish cartridge 16 may be removed from the sterile filling environment without compromising the sterility or container integrity of the drug container 18, fluid assembly pathway 20, or their individual components.

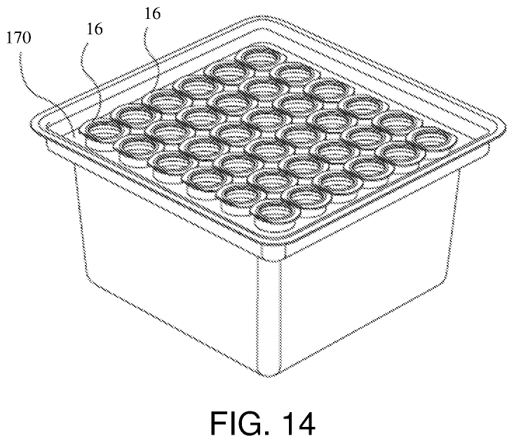

According to another aspect of the invention, embodiments of the present invention may enable the drug containers to be filled in standard fill-finish processes. In this regard, the fill-finish cartridges may utilize existing or standardized fill-finish equipment. A plurality of drug containers may be removably mounted, mated, inserted, or otherwise placed into a standard fill-finish tray such as illustrated in FIG. 14, for filling with pharmaceutical treatments. The flange 40a of the carrier 40 may assist in placement and handling of the carriers 40 and drug containers 18. The flange 40a may be configured to interface with a tray 170 which supports one or more fill-finish cartridges during the fill-finish process. The tray 170 may include one or more apertures, through which the body of the carrier 40 may be inserted, the diameter of the apertures being smaller than the outer diameter of the flange 40a. Hence, after inserting the body of the carrier 40 through the aperture, the flange 40a may rest upon, and be supported by, the tray 170. The fill-finish cartridges 116 may then be processed through the fill-finish process while supported by the tray 170. The drug containers 18 may be filled in automated assembly and drug filling lines and then sealed by insertion of a plunger seal into the proximal end of the drug container 18. Fill-finish trays of any configuration or capable of holding any number of containers may be utilized.

The fluid pathway components are connected such that the sterility of the fluid pathway from the fluid pathway connection, through the fluid conduit, to the needle insertion mechanism is maintained. These novel fill-finish cartridges of the present invention function to maintain the sterility of the fluid pathway assemblies and allow them to nest, mount, or otherwise be removably inserted into trays for standard fill-finish processes. As shown in FIG. 3, in such an embodiment, the carrier 40 may include an upper carrier 42 and a lower carrier 44. The drug container 18 being generally positioned within the upper carrier. The needle insertion mechanism, conduit, and fluid pathway connection being positioned generally within the lower carrier. The upper and lower carriers may, optionally, be composed of more than one separable component.

The carrier maintains the drug container 18, needle insertion mechanism 24, and fluid pathway connection 22 in position such that the central axis of the drug container 18 passes through both the needle insertion mechanism 24 and fluid pathway connection 22. This alignment allows the cartridge to be positioned in standard fill-finish trays, thereby minimizing the changes required to accommodate such a cartridge.

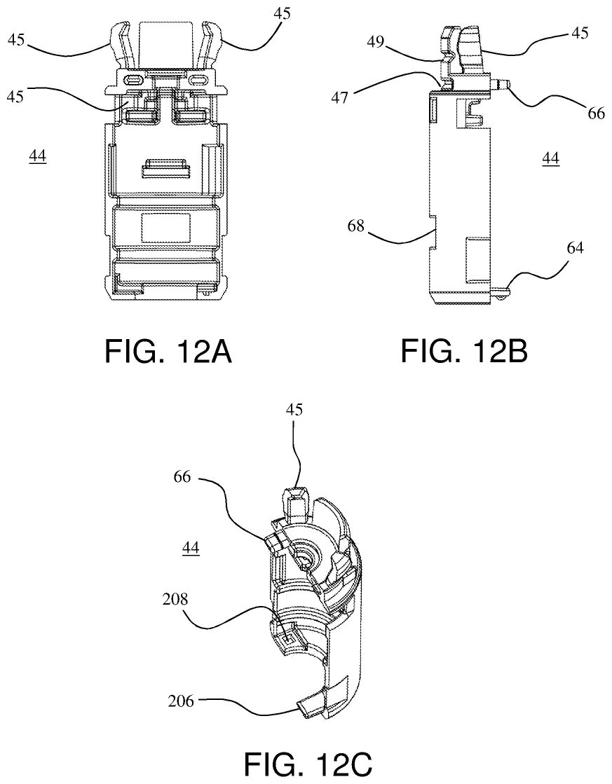

One embodiment of a fill-finish cartridge 16 is shown in FIGS. 3-11. The embodiment illustrated therein includes an upper carrier 42 and a lower carrier 44 which includes a first lower carrier 46 and second lower carrier 48. The lower carrier 44 is configured to engage and position needle insertion mechanism 24 and fluid pathway connection 22. In at least one embodiment, fluid pathway connection 22 and needle insertion mechanism 24 are fluidly coupled by fluid conduit 26. The fluid pathway connection 22 may be positioned within pocket 45 (shown in FIG. 12A), thereby restricting axial translation of fluid pathway connection 22. In some embodiments, limited motion of the fluid pathway connection 22 is permitted. This limited motion of fluid pathway connection 22 may allow it to be properly positioned with respect to drug container 18. At least one of the first lower carrier 46 and second lower carrier 48 may include one or more engagement arms 206 and the other portion may include one or more corresponding engagement windows 208 (shown in FIG. 12C). Connection of the engagement arms 206 and engagement windows 208 securely and removably connects the first lower carrier 46 and second lower carrier 48. In the embodiment shown in FIG. 12C, each of the first lower carrier and second lower carrier have both an engagement arm 206 and engagement window 208 configured to engage the corresponding feature on the other component. First lower carrier 46 and second lower carrier 48 may be further maintained in a connected position by the interaction of upper carrier 42 and lower carrier 44.

Lower carrier 44 may further be configured for engagement with upper carrier 42. For example, one of the lower carrier or the upper carrier may include one or more locking prongs configured to engage a corresponding connection recess on the opposite component. In the illustrated embodiment, upper carrier 42 includes one or more connection prongs 42j configured to engage corresponding connection recesses 47 of lower carrier 44. Connection prongs 42j may be configured to be relatively flexible portions which are configured to flex radially outward in response to contact with lower carrier 44. Upon alignment with connection recesses 47, connection prongs 42j are able to return toward their natural position and engage the connection recess 47. Connection prongs 42j and connection recess 47 may be configured such that rotation of upper carrier 42 with respect to lower carrier 44 causes the connection prongs 42j to disengage connection recess 47. This allows upper carrier 42 to be removed from lower carrier 44 after completion of the fill-finish process. Although the illustrated embodiment shows the connection prongs as an aspect of the upper carrier and the connection recess as an aspect of the lower carrier, it is also contemplated that this relationship may be reversed (i. e., the connection prongs are an aspect of the lower carrier and the connection recess is an aspect of the upper carrier).

In addition to connection recess 47, lower carrier 44 may also include one or more intermediate recesses 49, as shown in the illustrated embodiment. These intermediate recesses are configured for temporary engagement with connection prongs 42j during assembly of fill-finish cartridge 16, as will be described further herein.

Further, lower carrier 44 may include one or more features configured to engage drug container 18. For example, one or more portions of lower carrier 44 may also include one or more retention features 45. The retention features 45 may be configured to engage the neck 18d, crimp cap 62 and/or collar 18e of the drug container 18. Retention features 45 engage the drug container and restrict movement of the drug container relative to lower carrier 44 and, thereby, fluid pathway connection 22. As a result, a piercing member 22a may be positioned within a cavity 50a of a pierceable seal 50 of the drug container 18. Additionally, a portion of the fluid pathway connection 22 may form a sealing engagement with the pierceable seal 50. Thus, when sterilized, cavity 50a of pierceable seal 50, sealed by fluid pathway connection 22, forms a sterile volume. A portion of the piercing member 22a is disposed within the sterile volume, thereby maintaining the sterility of a portion of the piercing member 22a. Retention features 45 may be constructed to flex radially outward in response to contact with a portion of the drug container 18. When aligned with the neck 18d of the drug container 18, the retention features 45 may return toward their natural positions to engage the drug container 18. The upper carrier 42 may include apertures or reliefs which allow clearance for the retention features 45 to flex radially outward, for example, when connection prongs 42j are engaged with intermediate recesses 49.

Alternatively, retention features may be forced radially inwardly by interference with upper carrier 42. In such an embodiment, the natural position of the one or more retention features 45 may be such that the drug container 18 may pass within the retention features 45 without displacing them. When upper carrier 42 is placed in its final position, interference between upper carrier 42 and retention features 45 may cause them to flex radially inward in order to engage drug container 18.

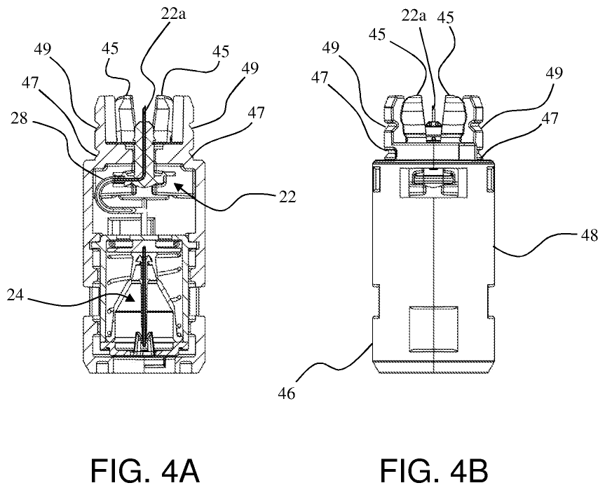

The various stages of assembly of one embodiment of a fill-finish cartridge are shown in FIGS. 4A-11 and additional aspects of the invention will be described with reference to these figures. As shown in FIG. 4A, fluid pathway assembly 20 is disposed within lower carrier 44. In this position, fluid pathway connection 22 is fluidly coupled to needle insertion mechanism 24 by fluid conduit 26. Fluid pathway connection 22 and needle insertion mechanism 24 are mechanically coupled by lower carrier 44, thereby maintaining the relative spatial relationship of these components. As seen in FIG. 4B, lower carrier 44 includes first lower carrier 46 and second lower carrier 48 which are connected together to enclose fluid pathway assembly 20. As seen in FIGS. 4A and 4B, piercing member 22a may extend from lower carrier 44 such that it may engage a seal of drug container 18, as will be described further herein.

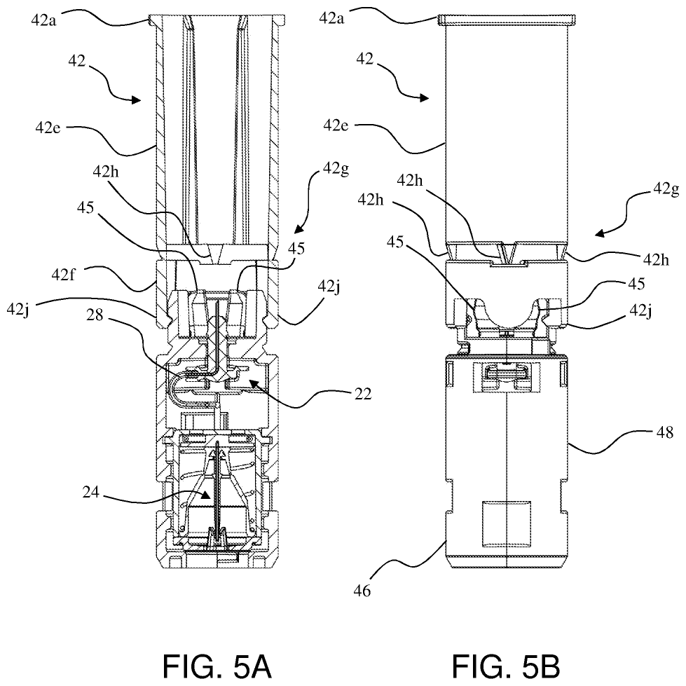

FIGS. 5A and 5B show fill-finish cartridge 16 after initial connection of upper carrier 42 to lower carrier 44. In this position, connection prongs 42j are engaged with intermediate recess 49. This connects upper carrier 42 to lower carrier 44 while allowing clearance such that retention features 45 may be in a position to allow passage of drug container 18. In this position, fill-finish cartridge 16 is prepared for connection of drug container 18.

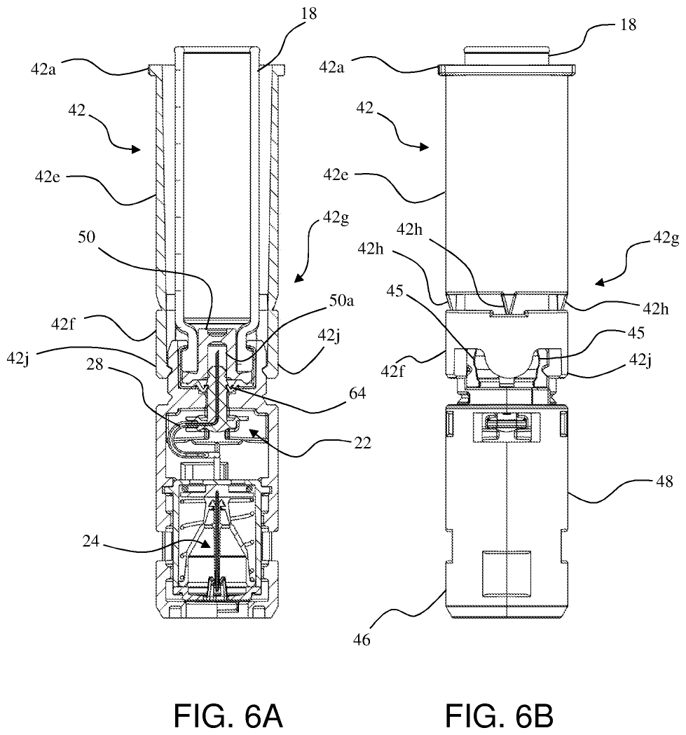

FIGS. 6A and 6B show fill-finish cartridge 16 with drug container 18 inserted. In this position, piercing member 22a is disposed within cavity 50a of pierceable seal 50. The engagement of retention features 45 with neck 18d and/or crimp cap 62 maintains the spatial relationship of drug container 18 and fluid pathway connector 22. Optionally, the drug container may include one or more hub retainers 64 which engage a portion of fluid pathway connector 22, thereby further restricting relative motion of the drug container and fluid pathway connector. One embodiment of this is shown in FIG. 6A. By allowing some movement of fluid pathway connection 22 within lower carrier 44, hub retainer 64 is able to engage fluid pathway connection 22 while allowing for tolerance variation. In other words, the spatial relationship of drug container 18 and fluid pathway connection 22 may be determined by hub retainer 64 and not by upper carrier 42 or lower carrier 44. This may ensure that at least a portion of fluid pathway connection 22 is in contact with pierceable seal 50 to maintain the sterility of piercing member 22a.

FIGS. 7A and 7B show fill-finish cartridge 16 with upper carrier 42 in a locked position. As can be seen by comparing FIG. 6A and FIG. 7A, upper carrier 42 is translated in the distal direction with respect to lower carrier 44. As a result, connection prongs 42j engage connection recess 47. In this position, upper carrier 42 prevents radially outward displacement of retention features 45 and locks retention features 45 in an engaged position. As a result, drug container 18 cannot be removed from fill-finish cartridge 16. In the configuration shown in FIGS. 7A and 7B, fill-finish cartridge 16 is prepared to be processed in a fill-finish process. Prior to filling, assembly 16 may be sterilized using any appropriate sterilization process. In one embodiment, fill-finish cartridge is sterilized using a gas sterilization process such as an ethylene oxide sterilization process. Upper carrier 42 and/or lower carrier 44 may include one or more windows to increase the efficacy of the sterilization process and to aid in assembly. After sterilization, fill-finish cartridge 16 may be placed in a tub or tray. Alternatively, the components of the fill-finish cartridge may be sterilized prior to assembly.

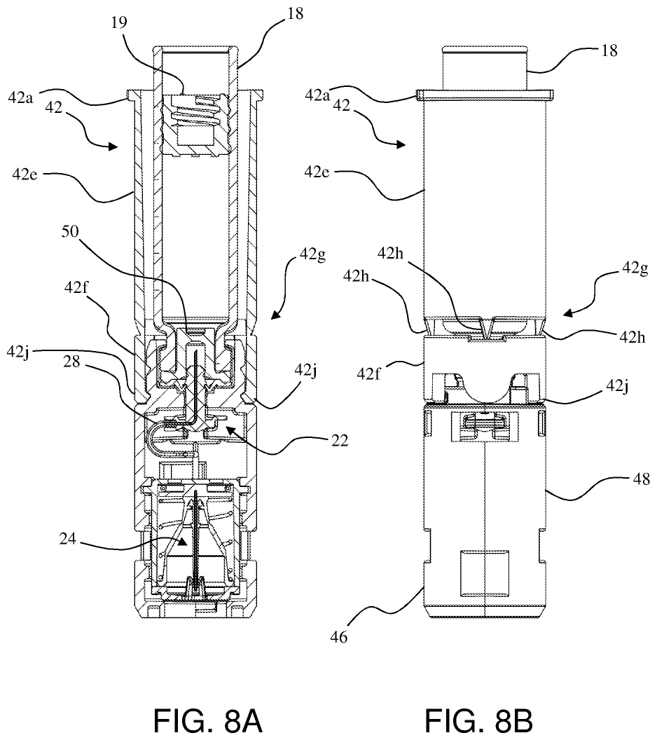

FIGS. 8A and 8B show the fill-finish cartridge 16 after the filling and stoppering process. Drug container 18 contains the filled medicament which has been filled through proximal opening 18a. Plunger seal 19 has been placed within drug container 18 near its open proximal end. Optionally, a vacuum stoppering process may be used to place the plunger seal.

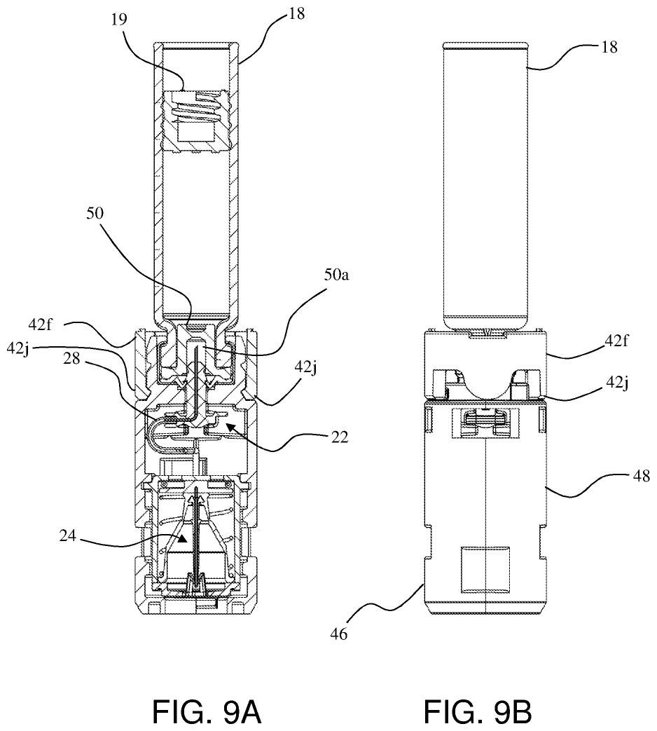

In some instances, it may be necessary to inspect the drug container and the drug contained therein. This inspection may include spinning the drug container in order to determine the presence of particles contained therein. In order to allow for an unobstructed view of the drug container, the upper carrier may be removed. Alternatively, as illustrated, upper carrier 42 may include tubular body 42e and locking sleeve 42f which are separable at junction 42g. This allows tubular body 42f to be removed after filling while locking sleeve 42f remains in place. With locking sleeve 42f in place, retention features 45 are not able to flex radially outward and they, therefore, securely retain drug container 18. This configuration is shown in FIGS. 9A and 9B. In the embodiment illustrated, junction 42g consists of frangible tabs 42h. Hence, rotation of tubular body 42f severs frangible tabs 42h and allows removal of tubular body 42f. Frangible tabs 42h may alternatively be severed in using other methods such as applying an axial force to tubular body 42e. While not illustrated, junction 42g may alternatively consist of any means of connection, such as a threaded connection, a press-fit connection, or any other means known to one skilled in the art.

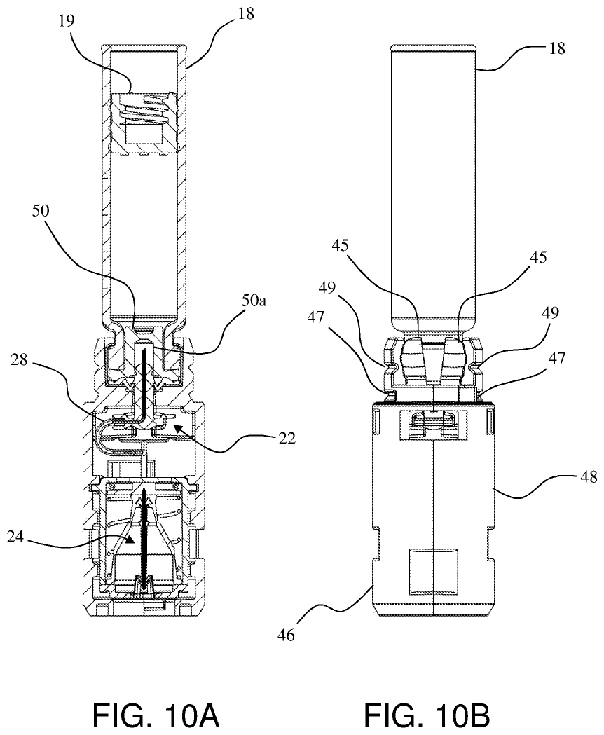

FIGS. 10A and 10B show fill-finish cartridge 16 after removal of locking sleeve 42f. In the embodiment illustrated, locking sleeve 42f is removed by rotation relative to lower carrier 44, thereby releasing connection prongs 42j from connection recess 47. Alternatively, locking sleeve 42f may consist of two or more separable components that may be disconnected in order to remove locking sleeve 42f from lower carrier 44.

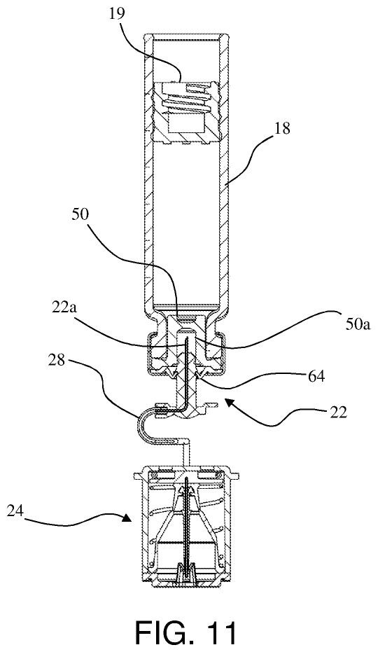

After removal of locking sleeve 42f, lower carrier 44 may be removed, thereby mechanically decoupling fluid pathway connector 22 from needle insertion mechanism 24. These components remain fluidly coupled by fluid conduit 26. Piercing member 22a remains disposed within cavity 50a, thereby maintaining the sterility of piercing member 22a. This may be in part a result of hub retainer 64 engaging fluid pathway connection 22 as shown in FIG. 11. With carrier 40 removed, drug container 18, fluid pathway connector 22, and needle insertion mechanism 24 may be placed in drug delivery device 10, as shown in FIG. 1. In the illustrated embodiment, carrier 40 is completely removed and is not included in drug delivery device 10. However, in other embodiments, one or more aspects of carrier 40 remain connected to one of drug container 18, fluid pathway connection 22, and/or needle insertion mechanism 24 when inserted into drug delivery device 10.

Additionally, one or more embodiments facilitate the use of vacuum stoppering to position the plunger seal within drug container 18. By so doing, the amount of entrapped air within the drug container may be significantly reduced. This may be accomplished with minimal or no modifications to standard fill-finish equipment.

To allow the use of vacuum stoppering equipment with flangeless drug containers, a sealing member (not shown) may be positioned between the drug container 18 and the carrier 40. The sealing member may be, for example, an elastomeric member such as an o-ring. The sealing member may be located at any position along the drug container 18 or, alternatively, the sealing member may be in contact with crimp cap 62. The carrier 40 or drug container 18 may include a groove, recess, or other feature within which the sealing member may be positioned. In at least one embodiment, a plurality of sealing members are positioned at multiple positions within the carrier, this may increase the efficacy of the sealing member. By providing a sealing member between drug container 18 and carrier 40, standard vacuum stoppering equipment may be used with the fill-finish cartridges of the present invention. The sealing element of the stopper placement tube may contact and seal against the flange of the carrier 40. The distal end of drug container 18 is closed by pierceable seal 50. The distal seal may be held in place by crimp cap 62. Because carrier 40 is in sealing engagement with drug container 18 due to the sealing member, a sealed volume is created within the carrier which includes the volume within the drug container 18. Hence, a vacuum may be pulled on this volume, thereby reducing the pressure within drug container 18 and allowing the plunger seal to be placed as previously described with regards to standard vacuum stoppering processes. This provides a simple and cost-effective method of placing the plunger seal within the drug container.

The sealing member may be a separate component such as an o-ring or, alternatively, the sealing member may be an integral portion of one or more components. For example, an elastomeric material could be co-molded with the carrier such that the elastomeric material is in contact with the drug container, crimp cap, or other component during the fill-finish process.

In an alternative embodiment, a radial seal at the proximal end of the drug container is used during a vacuum stoppering procedure. To facilitate the use of such a radial seal, the proximal end of drug container 18 may be configured to extend beyond carrier 40, as shown in FIG. 7B. During the stoppering procedure, the ring seal may engage this exposed portion of drug container 18 to allow for the creation of a vacuum.

In another embodiment, a method of use of processing a drug container may include the steps of: sealing the distal opening 18b of the drug container 18, inserting the drug container 18 into a carrier 40, inserting the carrier 40 and drug container 18 into a tray 170, and filling the drug container 18 with a drug. The method may further include the step of causing a retention feature 45 to engage a neck 18d and/or collar 18e of the drug container 18. The method also may include the step of inserting a seal into the proximal end of the drug container 18 to thereby seal the contents within the drug container 18. As would be appreciated by one having ordinary skill in the art, this filling and assembly process may be completed under vacuum and/or a sterile environment. The drug containers are configured such that they may readily be manufactured individually, or in a group, as is the case in a tray-based filling process.

In a further embodiment, the present invention relates to the method of using the fill-finish cartridge, which method may include the steps of: filling the drug container with a pharmaceutical drug; mounting the needle insertion mechanism to a first location of a drug delivery device; mounting the drug container to a second location of the drug delivery device; triggering the fluid pathway connection to pierce a permeable seal at a distal end of the drug container; triggering the needle insertion mechanism to insert a cannula into a patient; activating a drive mechanism to force the pharmaceutical drug out of the drug container and through the primary container connect, a fluid conduit, and the cannula of the needle insertion mechanism for drug dispersal into the patient. Upon completion of drug delivery, the method of use may further include the step of triggering the needle insertion mechanism to retract the cannula from the patient. The cannula may be a rigid needle, a flexible tube cannula, or a number of other known conduits for injection and/or drug delivery.

The novel drug containers and carriers of the present invention may provide substantial benefits in the marketplace. Embodiments of the present invention can readily be manufactured in a sterile environment, integrated into standard filling (e.g., fill-finish) process lines for aseptic filling of pharmaceutical treatments, and utilized for cost-effective assembly into drug delivery devices. Each of these advantages has substantial benefits over existing methodologies.

Additionally, the embodiments of the present invention allow for the utilization of standard fill-finish processes to fill the drug container. This greatly simplifies the manufacturing processes used to build drug delivery devices. Standard fill-finish processes utilize trays which hold multiple drug containers, such as syringes. The embodiments of the present invention enable a drug delivery device manufacturer, pharmaceutical company, or contract drug filler to fill the drug containers for infusion or injection pumps using the same standard fill-finish processes. These drug containers can be filled aseptically, as is common industry practice, in a cost-efficient manner.

It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

The use of the terms “a” and “an” and “the” and “at least one” and similar referents in the context of describing the invention are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Moreover, any combination of the above-described elements in all possible variations thereof is encompassed by the disclosure unless otherwise indicated herein or otherwise clearly contradicted by context.

1. A cartridge for use in a medicament filling and finishing process, the cartridge comprising:
a drug container defining a longitudinal axis,
a needle insertion mechanism including a needle;
a fluid pathway connection coupled to the drug container;
a flexible, sterile fluid conduit fluidly coupled to the needle insertion mechanism and to the fluid pathway connection; and
a carrier, the carrier including an upper carrier and a lower carrier;
wherein the lower carrier includes one or more retention features engaged with the drug container to removably mechanically couple the drug container, the fluid pathway connection, and the needle insertion mechanism.

2. The cartridge of claim 1, wherein the upper carrier includes a junction configured to allow the separation of a tubular body of the upper carrier from a retention sleeve of the upper carrier.

3. The cartridge of claim 2, wherein the junction includes one or more frangible tabs.

4. The cartridge of claim 1, wherein the lower carrier defines a lower carrier cavity, the needle insertion mechanism and the fluid pathway connection disposed at least partially within the lower carrier cavity.

5. The cartridge of claim 1, wherein the upper carrier defines an upper carrier cavity, the drug container is disposed at least partially within the upper carrier cavity.

6. The cartridge of claim 1, wherein the upper carrier includes one or more connection prongs and the lower carrier includes one or more connection recesses, the connection prongs disposed at least partially within the connection recesses to mechanically couple the upper carrier and the lower carrier.

7. The cartridge of claim 6, wherein the upper carrier is decoupled from the lower carrier by rotation of the upper carrier relative to the lower carrier which releases the connection prongs from the connection recesses.

8. The cartridge of claim 1, wherein the lower carrier includes a first lower carrier and a second lower carrier, the first lower carrier being connected to the second lower carrier to removably mechanically couple the fluid pathway connection and the needle insertion mechanism.

9. (canceled)

10. (canceled)

11. The cartridge of claim 1, further comprising a fluid contained within the drug container and a plunger seal disposed within the drug container adjacent a proximal opening of the drug container.

12. The cartridge of claim 1, wherein the upper carrier includes a flange at a proximal end of the upper carrier.

13. The cartridge of claim 1, wherein the one or more retention features are engaged with a neck of the drug container.

14. The cartridge of claim 1, wherein the one or more retention features are engaged with a crimp collar of the drug container.

15. A cartridge for use in a medicament filling and finishing process, the cartridge comprising:
a drug container defining a longitudinal axis,
a needle insertion mechanism including a needle;
a fluid pathway connection coupled to the drug container;
a flexible, sterile fluid conduit fluidly coupled to the needle insertion mechanism and to the fluid pathway connection; and
a carrier, the carrier including an upper carrier and a lower carrier;
wherein the drug container, the fluid pathway connection, and the needle insertion mechanism are removably mechanically coupled by the carrier and wherein the upper carrier includes a junction configured to allow the separation of a tubular body of the upper carrier from a retention sleeve of the upper carrier.

16. The cartridge of claim 15, wherein the junction includes one or more frangible tabs.

17. (canceled)

18. (canceled)

19. The cartridge of claim 15, wherein the lower carrier defines a lower carrier cavity, the needle insertion mechanism and the fluid pathway connection are disposed at least partially within the lower carrier cavity.

20. The cartridge of claim 15, wherein the upper carrier defines an upper carrier cavity, the drug container disposed at least partially within the upper carrier cavity.

21. The cartridge of claim 15, wherein the upper carrier includes one or more connection prongs and the lower carrier includes one or more connection recesses, the connection prongs disposed at least partially within the connection recesses to mechanically couple the upper carrier and the lower carrier.

22. (canceled)

23. The cartridge of claim 15, wherein the lower carrier includes a first lower carrier and a second lower carrier, the first lower carrier being connected to the second lower carrier to removably mechanically couple the fluid pathway connection and the needle insertion mechanism.

24. (canceled)

25. (canceled)

26. The cartridge of claim 15, further comprising a fluid contained within the drug container and a plunger seal disposed within the drug container adjacent a proximal opening of the drug container.

27. The cartridge of claim 15, wherein the upper carrier includes a flange at a proximal end of the upper carrier.

28. A method of constructing a cartridge for a medicament filling and finishing process, the method comprising:
fluidly coupling a fluid pathway connection to a needle insertion mechanism;
removably mechanically coupling the fluid pathway connection and the needle insertion mechanism by disposing the fluid pathway connection and the needle insertion mechanism in a lower carrier cavity of a lower carrier;
connecting an upper carrier to the lower carrier; and
disposing a drug container within an upper carrier cavity of the upper carrier and engaging the drug container with one or more retention features of the lower carrier.

29.-34. (canceled)

35. A method of deconstructing a cartridge of claim 15 for a medicament filling and finishing process, the method comprising:
decoupling the tubular body of the upper carrier from a retention sleeve of the upper carrier;
disconnecting the retention sleeve of the upper carrier from a lower carrier; and
mechanically decoupling a fluid pathway connection from a needle insertion mechanism by removing the fluid pathway connection and the needle insertion mechanism from a lower carrier cavity of the lower carrier.

36.-38 (canceled)
